(19) United States
(12) Patent Application Publication
(10) Pub. No.: US 2025/0205234 A1
(43) Pub. Date: Jun. 26, 2025

(54) ANTI-TUBERCULAR COMPOSITIONS

(71) Applicants: Nanyang Technological University, Singapore (SG); Agency for Science, Technology and Research, Singapore (SG)

(72) Inventors: Gerhard GRÜBER, Singapore (SG); Roderick Wayland BATES, Singapore (SG); Shuyi Pearly NG, Singapore (SG)

(21) Appl. No.: 18/851,975

(22) PCT Filed: Mar. 31, 2023
(86) PCT No.: PCT/SG2023/050218

§ 371 Date: Sep. 27, 2024

(30) Foreign Application Priority Data
Apr. 1, 2022 (SG) 10202203339W

(51) Int. Cl. A61K 31/506; A61K 31/4545; A61K 31/4709; A61K 31/498; A61P 31/04; A61P 31/06

(52) U.S. Cl. CPC A61K 31/506; A61K 31/4545; A61K 31/4709; A61K 31/498; A61P 31/04; A61P 31/06

(57) ABSTRACT

Disclosed herein is a combination that includes a F1F0-ATP synthase inhibitor or a pharmaceutically acceptable salt or solvate thereof that selectively binds to the F1 domain of the F1F0-ATP synthase in combination with one or more of the compounds selected from: (a) an NADH dehydrogenase inhibitor or a pharmaceutically acceptable salt or solvate thereof; (b) a cytochrome-bcc:aa3 inhibitor or a pharmaceutically acceptable salt or solvate thereof; and (c) a F1F0-ATP synthase inhibitor or a pharmaceutically acceptable salt or solvate thereof that selectively binds to the F0 domain of the F1F0-ATP synthase.

### FIELD OF INVENTION

The present invention generally relates to anti-tubercular drugs and more particularly relates to anti-tubercular compositions for enhanced whole ATP synthesis inhibition and anti-tuberculosis activity.

### BACKGROUND

The listing or discussion of a prior-published document in this specification should not necessarily be taken as an acknowledgement that the document is part of the state of the art or is common general knowledge.

Tuberculosis (TB) is one of the major bacteria-caused infectious diseases that resulted in 1.5 million deaths in 2020, which for the first time in over a decade has increased according to the World Health Organization's 2021 Global TB report (WHO. Global Tuberculosis Report 2011; ISBN 978-92-4-003702-1; World Health Organization: Geneva, 2021). Multidrug resistant-(MDR) and extensively drug-resistant (XDR) bacterial strains of Mycobacterium tuberculosis (Mtb) which causes TB are spreading worldwide, causing major global health issues. Drug tolerance of Mtb is proposed to be due to the ability of the pathogen to enter a metabolically quiescent state, in which it is phenotypically tolerant to drug challenge with conventional chemotherapeutics (Gomez, J. E. & Mckinney, J. D. M., Tuberculosis (Edinb) 2004, 84, 29-44). In 2012, the anti-tuberculosis diarylquinoline bedaquiline (BDQ) was approved. BDQ kills with delay metabolically quiescent cells by targeting the mycobacterial F₁F₀ ATP synthase, and brought oxidative phosphorylation (OXPHOS) and the electron transport chain (ETC) complexes (FIG. 1) into the focus of anti-TB drug development. However, the successful advancement of this drug has been overshadowed by the development of clinical resistance, along with a very long terminal half-life, leading to concerns regarding tissue accumulation, and a study showing BDQ-inhibition of mitochondrial F-ATP synthase in human HEK293S cell mitoplasts (Luo, M. et al., Communications Biol. 2020, 3, 452).

The mycobacterial F₁F₀ ATP synthase which is a latent ATPase and essential for proper growth and colony formation, consists of the subunits α₃:β₃:γ:ε:b-δ:b′:a:c₉. Its H⁺-translocating F_{O} domain (subunits a:c₉) uses the proton motive force (PMF), generated by the ETC complexes NADH dehydrogenase (in case of NDH-1) and cytochrome bcc:aa₃ (cyt-bcc:aa₃), to drive rotation of the central stalk subunits γ-ε. The latter causes sequential binding, entrapment and phosphorylation of ADP to ATP within the nucleotide-binding and catalytic α₃:β₃-headpiece. The peripheral stalk subunits b-δ:b′ smoothen transmission of power between the rotary c-ring and the α₃:β₃:γ:ε domain.

Mycobacterial specific modifications of the F-ATP synthase subunits α, δ and γ, including a C-terminal elongation, an inserted domain or a 12-14 amino acids extra loop, respectively, have been described as regulative or essential features for catalysis. For example, the mycobacterial extra loop of subunit γ, absent in the human homologue and other prokaryotes, is important for ATP synthesis as well as ATP hydrolysis and ATP-driven proton pumping regulation, and has been identified as a new drug target. The most recent M. smegmatis F-ATP synthase cryo-electron microscopy (EM) structure shows a conformation in which one of the polar γ-loop residues forms a salt bridge with an arginine residue of the peripheral stalk subunit b′ during rotation (Montgomerya, M. G. et al., Proc. Natl. Acad. Sci. U.S.A 2021, 118, e2111899118).

Therefore, there exists a need for new compositions for specific targeting of the mycobacterial F-ATP synthase, and anti-TB potency in macrophages.

### SUMMARY OF INVENTION

Aspects and embodiments of the invention will be discussed by reference to the following numbered clauses.
1. A combination comprising:
(i) a F₁F₀-ATP synthase inhibitor or a pharmaceutically acceptable salt or solvate thereof that selectively binds to the F₁ domain of the F₁F₀-ATP synthase; and one or more of the compounds selected from:
(ii) an NADH dehydrogenase inhibitor or a pharmaceutically acceptable salt or solvate thereof;
(iii) a cytochrome-bcc:aa₃ inhibitor or a pharmaceutically acceptable salt or solvate thereof; and
(iv) a F₁F₀-ATP synthase inhibitor or a pharmaceutically acceptable salt or solvate thereof that selectively binds to the F₀ domain of the F₁F₀-ATP synthase, provided that the F₁F₀-ATP synthase inhibitor or a pharmaceutically acceptable salt or solvate thereof that selectively binds to the F₀ domain of the F₁F₀-ATP synthase is not diarylquinoline bedaquiline.
2. The combination according to Clause 1, wherein the F₁F₀-ATP synthase inhibitor that selectively binds to the F₁ domain of the F₁F₀-ATP synthase is a compound of formula Ia or Ib:

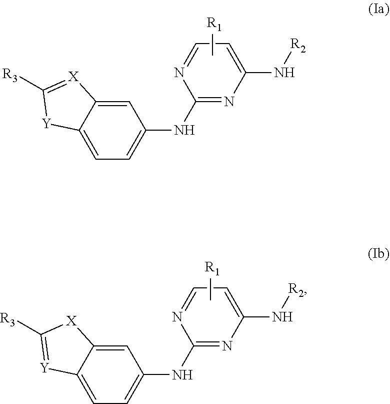

where:
R₁ is hydrogen or a methyl group;
R₂ is an unsubstituted or a substituted alkyl group;
R₃ is an aryl group or a heteroaryl group that is unsubstituted or substituted by one or more groups selected from halogen, alkyl or alkoxy; and,
In Formula Ia, X is CH or N and Y is NH, S or O, or,
in Formula Ib, X is NH, S or O and Y is CH or N, or a pharmaceutically acceptable salt or solvate thereof.
3. The combination according to Clause 2, wherein:
   in Formula Ia, X is N and Y is NH; or
   in Formula Ib, X is NH and Y is N.
4. The combination according to Clause 2 or Clause 3, wherein R₁ is a methyl group at the 6-position of the pyrimidine ring.
5. The combination according to any one of Clauses 2 to 4, wherein R₂ is an ethyl group or a —CH₂COOCH₂CH₃ group.
6. The combination according to any one of Clauses 2 to 5, wherein R₃ is an aryl group, optionally wherein the aryl group is phenyl.
7. The combination according to Clause 6, wherein the aryl group is substituted by one or more halogen atoms.
8. The combination according to any one of the preceding clauses, wherein the F₁F₀-ATP synthase inhibitor or a pharmaceutically acceptable salt or solvate thereof that selectively binds to the F₁ domain of the F₁F₀-ATP synthase is selected from the group consisting of:

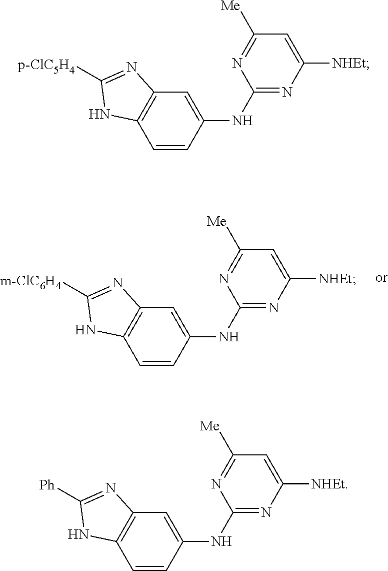

9. The combination according to any one of the preceding clauses, wherein the combination comprises a F₁F₀-ATP synthase inhibitor or a pharmaceutically acceptable salt or solvate thereof that selectively binds to the F₁ domain of the F₁F₀-ATP synthase and an NADH dehydrogenase inhibitor or a pharmaceutically acceptable salt or solvate thereof.
10. The combination according to any one of the preceding clauses, wherein the combination comprises a F₁F₀-ATP synthase inhibitor or a pharmaceutically acceptable salt or solvate thereof that selectively binds to the F₁ domain of the F₁F₀-ATP synthase and a cytochrome-bcc:aa₃ inhibitor or a pharmaceutically acceptable salt or solvate thereof.
11. The combination according to any one of the preceding clauses, wherein the combination comprises a F₁F₀-ATP synthase inhibitor or a pharmaceutically acceptable salt or solvate thereof that selectively binds to the F₁ domain of the F₁F₀-ATP synthase and a F₁F₀-ATP synthase inhibitor or a pharmaceutically acceptable salt or solvate thereof that selectively binds to the F₀ domain of the F₁F₀-ATP synthase.
12. The combination according to any one of Clauses 1 to 8, wherein the combination comprises a F₁F₀-ATP synthase inhibitor or a pharmaceutically acceptable salt or solvate thereof that selectively binds to the F₁ domain of the F₁F₀-ATP synthase, an NADH dehydrogenase inhibitor or a pharmaceutically acceptable salt or solvate thereof and a cytochrome-bcc:aa₃ inhibitor or a pharmaceutically acceptable salt or solvate thereof.
13. The combination according to any one of Clauses 1 to 8, wherein the combination comprises a F₁F₀-ATP synthase inhibitor or a pharmaceutically acceptable salt or solvate thereof that selectively binds to the F₁ domain of the F₁F₀-ATP synthase, an NADH dehydrogenase inhibitor or a pharmaceutically acceptable salt or solvate thereof and a F₁F₀-ATP synthase inhibitor or a pharmaceutically acceptable salt or solvate thereof that selectively binds to the F₀ domain of the F₁F₀-ATP synthase.
14. The combination according to any one of Clauses 1 to 8, wherein the combination comprises a F₁F₀-ATP synthase inhibitor or a pharmaceutically acceptable salt or solvate thereof that selectively binds to the F₁ domain of the F₁F₀-ATP synthase, a cytochrome-bcc:aa₃ inhibitor or a pharmaceutically acceptable salt or solvate thereof; and a F₁F₀-ATP synthase inhibitor or a pharmaceutically acceptable salt or solvate thereof that selectively binds to the F₀ domain of the F₁F₀-ATP synthase.
15. The combination according to any one of Clauses 1 to 8, wherein the combination comprises a F₁F₀-ATP synthase inhibitor or a pharmaceutically acceptable salt or solvate thereof that selectively binds to the F₁ domain of the F₁F₀-ATP synthase, an NADH dehydrogenase inhibitor or a pharmaceutically acceptable salt or solvate thereof, a cytochrome-bcc:aa₃ inhibitor or a pharmaceutically acceptable salt or solvate thereof, and a F₁F₀-ATP synthase inhibitor or a pharmaceutically acceptable salt or solvate thereof that selectively binds to the F₀ domain of the F₁F₀-ATP synthase.
16. The combination according to any one of Clauses 1 to 9, 12, 13 and 15, wherein the NADH dehydrogenase inhibitor or a pharmaceutically acceptable salt or solvate thereof is clofazimine.
17. The combination according to any one of Clauses 1 to 8, 10, 12, 14 and 15, wherein the cytochrome-bcc:aa₃ inhibitor or a pharmaceutically acceptable salt or solvate thereof is Q203:

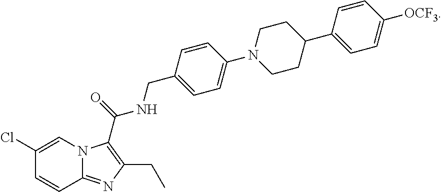 [SMILES: CCc1nc2ccc(Cl)cn2c1C(=O)NCc1ccc(N2CCC(c3ccc(OC(F)(F)F)cc3)CC2)cc1]

18. The combination according to any one of Clauses 1 to 8, 11, and 13 to 15, wherein the F₁F₀-ATP synthase inhibitor or a pharmaceutically acceptable salt or solvate thereof that selectively binds to the F₀ domain of the F₁F₀-ATP synthase is TBAJ876:

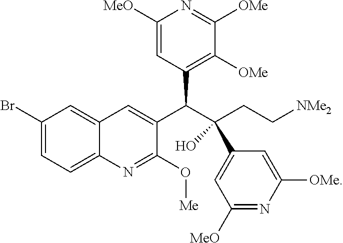 [SMILES: COc1cc([C@](O)(CCN(C)C)[C@@H](c2cc3cc(Br)ccc3nc2OC)c2cc(OC)nc(OC)c2OC)cc(OC)n1]

19. A pharmaceutical composition comprising a pharmaceutically acceptable diluent or carrier and a combination according to any one of Clauses 1 to 18.
20. A kit of parts comprising a combination according to any one of Clauses 1 to 18, and optionally, instructions for treating a patient.
21. Use of a combination according to any one of Clauses 1 to 18, a composition according to Clause 19 or a kit according to Clause 20 in medicine.
22. Use of a combination according to any one of Clauses 1 to 18, a composition according to Clause 19 or a kit of parts according to Clause 20 in the manufacture of a medicament to treat a bacterial infection.
23. A method of treating a bacterial infection comprising the steps of administering to a subject in need thereof a pharmaceutically acceptable amount of each component of the combination as described in any one of Clauses 1 to 18, wherein each component of the combination is administered sequentially, simultaneously or concomitantly with the other components.
24. A method of treating a bacterial infection comprising the steps of administering to a subject in need thereof a pharmaceutically acceptable amount of the composition as described in Clause 19.
25. A method of treating a bacterial infection comprising the steps of administering to a subject in need thereof a pharmaceutically acceptable amount of each component of the kit of parts as described in Clause 20 to a subject in need thereof, wherein each component of the kit of parts is administered sequentially, simultaneously or concomitantly with respect to each of the other components.
26. A combination according to any one of Clauses 1 to 18, a composition according to Clause 19 or a kit of parts according to Clause 20 for use in the treatment of a bacterial infection.
27. The use according to Clause 22, the method according to any one of Clauses 23 to 25, the combination for use according to Clause 26, the composition for use according to Clause 26, or the kit of parts for use according to Clause 26, wherein the bacterial infection is tuberculosis.

### DRAWINGS

FIG. 1 depicts the enzyme composition and inhibitors of the mycobacterial ETC and F-ATP synthase. The cryo-EM structures of the M. smegmatis (Ms) fumarate reductase (PDB ID: 6LUM), cyt-bc1:aa3 supercomplex (PDB ID: 7RH5), the Mtb cyt-bd (PDB ID: 7NKZ, and the Ms F-ATP synthase (PDB ID 7NJK) are shown.

FIG. 2 depicts the (A) growth inhibition of M. bovis BCG cells by GaMF1.39. Three independent experiments were carried out, each with three technical replicates (B) Intracellular ATP synthesis of M. bovis BCG cells by GaMF1.39 (inverted triangles) in comparison to BDQ (open circles). (C) Inhibition of ATP synthesis by GaMF1.39 in inside-out membrane vesicles (IMVs) using the electron donor succinate (inverted triangles). BDQ was used as a control BDQ (open circles). ****: P<0.0001, statistical analysis was carried out for the experiment using one sample t and Wilcoxon test. (D) GaMF1.39 did also not affect oxygen consumption in the slow grower M. bovis BCG over a 96-h period. Thioridazine was used as positive control and 7H9 media with dimethyl sulfoxide (DMSO) as solvent served as a blank/negative control. (E) GaMF1.39 does not function as an uncoupler. Effects of GaMF1.39 on the transmembrane pH gradient of mycobacterial IMVs. 1 mM of GaMF1.39 does not alter the quenching of fluorescence of the pH-sensitive fluorophore ACMA. At the beginning of the experiments, 2 mM NADH, an electron donor, was added to the vesicle samples. The IMVs oxidized NADH and pumped protons to generate the transmembrane pH gradient, visualized as quenching of fluorescence; Uncoupler SF6847 (1 μM) were added at the end of each experiment as a positive control to collapse the transmembrane pH gradient. The vertical dotted lines indicate the time points at which NADH, GaMF1.39 or SF6847 was added. Solid black line shows the profile in the presence of GamF1.39 and the dotted black line shows the profile in the presence of DMSO as a control. (F) Inhibition of ATP synthesis by BDQ (open squares) and GaMF1.39 (inverted triangles) on M. smegmatis 166M c-subunit mutant IMVs with NADH as substrate. The effect of BDQ on wild-type IMV'S (open circles) are shown as control. (G) Effect of GaMF1.39 on ATP synthesis of reconstituted M. smegmatis F-ATP synthase. The inhibitory effect of GaMF1.39 (90 and 180 nM) of M. smegmatis F-ATP synthase, which was reconstituted into proteoliposomes. BDQ at an IC₅₀ concentration of 4 nM was used in comparison. In the no compound sample (DMSO), an end-concentration of 1.25% (v/v) was added, to see whether DMSO has an effect on the ATP synthesis rate. The result represents two independent experiments (n=2), performed at least in triplicates. ****: P<0.0001, statistical analysis was carried out for the experiment using the ordinary one way ANOVA test.

FIG. 3 depicts the initial six days of GaMF1.39 kill kinetics against M. bovis (BCG). The bacteria were grown in liquid culture (LBT) (A) in the presence of the indicated concentrations of GaMF1.39 up to 6 days. (B) CFU was calculated by plating the culture on 7H10 agar plates. (C) Initial five days of GaMF1.39 kill kinetics against M. smegmatis mc² 155. plates. *: P<0.05, statistical analysis was carried out for the experiment using the ordinary one way ANOVA test.

FIG. 4 depicts the (A) initial six days of untreated and GaMF1.39 kill kinetics against M. bovis (BCG). The bacteria were grown in liquid culture (LBT) in the presence of the indicated concentrations of GaMF1.39 up to 6 days. CFU was calculated by plating the culture on 7H10 agar plates. *: P<0.05, statistical analysis was carried out for the experiment using the ordinary one way ANOVA test. (B) Intracellular efficacy of GaMF1.39 on M. tuberculosis H37Rv-infected THP-1 cells. Macrophages were infected with M. tuberculosis H37Rv and treated with GaMF1.39 (3 μM, 9 μM), BDQ (2 μM) and DMSO. Experiments were repeated 3 times in triplicates, and analyzed with the one-way ANOVA test, *, P≤0.05; **, P≤0.01; ***, P≤0.001; ****, P≤0.0001. (C) Toxicity test of GaMF1.39 in zebrafish embryos. 48 hpf embryos were exposed to 3 and 9 M of GaMF1.39 dissolved in E3 medium for 5 days. Survival plot shows the number of live embryos/larvae upon exposure to GaMF1.39. DMSO was used as vehicle control. The inset shows the phenotype of the embryos exposed to DMSO and GaMF1.39. Two independent experiments were carried out, each with three technical replicates.

FIG. 5 depicts the activity of 1-3 μM of BDQ and 3-9 μM of GaMF1.39 against 3 h pre-formed biofilm of P. aeruginosa PAO1 wild type (WT) (A, C) and E. coli UTI 189 (B, D). Treatment was carried out in 1× phosphate buffered saline (PBS) media (A, B) or M9 glucose media (C, D). Both planktonic and biofilm cells were collected from the same sample well, with planktonic cells (circles) referring to suspended cells present in treatment buffer prior to washing and collection of biofilm cells (open triangles). No significant differences were observed between GaMF1.39, untreated controls and the respective BDQ controls. At least two independent experiments were carried out, each with two technical replicates. Each data point represents averaged data of an independent experiment and the line represents the means of all data.

FIG. 6 depicts the enhanced potency of GaMF1.39 in combination with the antibiotics clofazimine (CFZ), Q203 or TBAJ-876. (A) GaMF1.39 (open circle) susceptibility of M. bovis BCG in combination with indicated concentrations of CFZ. (B) The inhibitory effect of GaMF1.39 in combination with Q203 (open diamond). (C) TBAJ-876, which was synthesized according to Sarathy, J. P. et al., Antimicrob. Agents Chemother. 2020, 64, e02404-19, increases the potency of GaMF1.39 growth inhibition of M. bovis BCG. (D) Whole cell ATP depletion of the combinations GaMF1.39+CFZ (0.5 μM), GaMF1.39+Q203 (100 nM) or GaMF1.39+TBAJ-876 (4 nM) in comparison to GaMF1.39 alone. ****: P<0.0001, statistical analysis was carried out for all the above experiments using the ordinary one way ANOVA Bartlett's test.

FIG. 7 depicts the isobolograms of the combination of GaMF1.39 and Q203 (A) or GaMF1.39 and Clofazamine (B) against M. bovis BCG. The dash line with triangle markers indicates ideal isobole, where drugs act in synergy and independently. The dash line indicates ideal isobole, where drugs act additively and in partial synergy.

FIG. 8 depicts the M. bovis BCG cultures grown for killing kinetics experiment in the absence of any drug, in the presence of GaMF1.39 (10×MIC₅₀) and GaMF1.39 (10×MIC₅₀)+Q203 (100 nM). The picture of the flasks was taken at t=10 days, showing the killing potency of GaMF1.39 and GaMF1.39+Q203.

### DESCRIPTION

It has been surprisingly found that a F₁F₀-ATP synthase inhibitor or a pharmaceutically acceptable salt or solvate thereof that selectively binds to the F₁ domain of the F₁F₀-ATP synthase can synergistically be used in combination with one or more other active agents to treat a bacterial infection. For example, it has been found that the combination of a F₁F₀-ATP synthase inhibitor or a pharmaceutically acceptable salt or solvate thereof that selectively binds to the F₁ domain of the F₁F₀-ATP synthase (e.g. GaMF1.39) with an NADH Dehydrogenase inhibitor (e.g. CFZ), a cyt-bcc:aa₃ inhibitor (e.g. Q203), and/or a F₁F₀-ATP synthase inhibitor or a pharmaceutically acceptable salt or solvate thereof that selectively binds to the F₀ domain of the F₁F₀-ATP synthase (e.g. TBAJ876) showed enhanced whole ATP synthesis inhibition and anti-tuberculosis activity.

Thus, in a first aspect of the invention, there is provided a combination comprising:
(i) a F₁F₀-ATP synthase inhibitor or a pharmaceutically acceptable salt or solvate thereof that selectively binds to the F₁ domain of the F₁F₀-ATP synthase; and one or more of the compounds selected from:
(ii) an NADH dehydrogenase inhibitor or a pharmaceutically acceptable salt or solvate thereof;
(iii) a cytochrome-bcc:aa₃ inhibitor or a pharmaceutically acceptable salt or solvate thereof; and
(iv) a F₁F₀-ATP synthase inhibitor or a pharmaceutically acceptable salt or solvate thereof that selectively binds to the F₀ domain of the F₁F₀-ATP synthase, provided that the F₁F₀-ATP synthase inhibitor or a pharmaceutically acceptable salt or solvate thereof that selectively binds to the F₀ domain of the F₁F₀-ATP synthase is not diarylquinoline bedaquiline.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions.

As will be appreciated, the compounds of (i) differ from the compounds of (iv) in respect of the domain of the F₁F₀-ATP synthase that they bind to.

Any suitable F₁F₀-ATP synthase inhibitor or a pharmaceutically acceptable salt or solvate thereof that selectively binds to the F₁ domain of the F₁F₀-ATP synthase may be used herein.

In particular embodiments of the invention that may be mentioned herein, the F₁F₀-ATP synthase inhibitor that selectively binds to the F₁ domain of the F₁F₀-ATP synthase may be a compound of formula Ia or Ib:

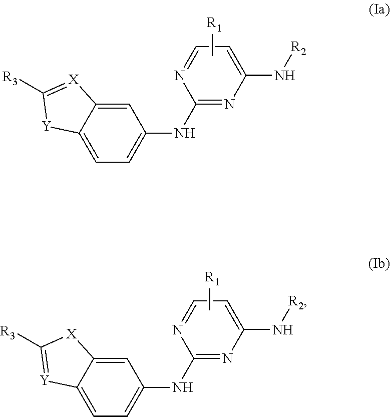

where:
R₁ is hydrogen or a methyl group;
R₂ is an unsubstituted or a substituted alkyl group;
R₃ is an aryl group or a heteroaryl group that is unsubstituted or substituted by one or more groups selected from halogen, alkyl or alkoxy; and,
In Formula Ia, X is CH or N and Y is NH, S or O, or,
in Formula Ib, X is NH, S or O and Y is CH or N, or a pharmaceutically acceptable salt or solvate thereof.

For the absolute avoidance of doubt, the F₁F₀-ATP synthase inhibitor or a pharmaceutically acceptable salt or solvate thereof that selectively binds to the F₀ domain of the F₁F₀-ATP synthase is not a compound of formula Ia or Ib, nor is it diarylquinoline bedaquiline. Any other F₁F₀-ATP synthase inhibitor or a pharmaceutically acceptable salt or solvate thereof that selectively binds to the F₀ domain of the F₁F₀-ATP synthase may be used herein. For example, the F₁F₀-ATP synthase inhibitor or a pharmaceutically acceptable salt or solvate thereof that selectively binds to the F₀ domain of the F₁F₀-ATP synthase may be TBAJ876:

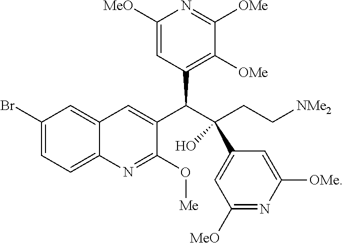 [SMILES: COc1cc([C@](O)(CCN(C)C)[C@@H](c2cc3cc(Br)ccc3nc2OC)c2cc(OC)nc(OC)c2OC)cc(OC)n1]

References herein (in any aspect or embodiment of the invention) to compounds of formula Ia and formula Ib include references to such compounds per se, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a compound of formula Ia or Ib with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula Ia or Ib in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula Ia or Ib are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent).

Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

“Pharmaceutically functional derivatives” of compounds of formula Ia or Ib as defined herein includes ester derivatives and/or derivatives that have, or provide for, the same biological function and/or activity as any relevant compound of the invention. Thus, for the purposes of this invention, the term also includes prodrugs of compounds of formula Ia or Ib.

The term “prodrug” of a relevant compound of formula Ia or Ib includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

Prodrugs of compounds of formula Ia or Ib may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula Ia or Ib wherein a hydroxyl, amino, sulfhydryl, carboxyl or carbonyl group in a compound of formula Ia or Ib is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. I-92, Elsevier, New York-Oxford (1985).

Compounds of formula Ia or Ib, as well as pharmaceutically acceptable salts, solvates and pharmaceutically functional derivatives of such compounds are, for the sake of brevity, hereinafter referred to together as the “compounds of formula Ia or Ib”.

Compounds of formula Ia or Ib may contain double bonds and may thus exist as E (entgegen) and Z (zusammen) geometric isomers about each individual double bond. All such isomers and mixtures thereof are included within the scope of the invention.

Compounds of formula Ia or Ib may exist as regioisomers and may also exhibit tautomerism. All tautomeric forms and mixtures thereof are included within the scope of the invention.

Compounds of formula Ia or Ib may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

The term “halo”, when used herein, includes references to fluoro, chloro, bromo and iodo.

Unless otherwise stated, the term “aryl” when used herein includes C₆₋₁₄ (such as C₆₋₁₀) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Embodiments of the invention that may be mentioned include those in which aryl is phenyl.

Unless otherwise stated, the term “alkyl” refers to an unbranched or branched, acyclic or cyclic, saturated or unsaturated (so forming, for example, an alkenyl or alkynyl) hydrocarbyl radical, which may be substituted or unsubstituted (with, for example, one or more halo atoms). Where the term “alkyl” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅₋₁₀ (e.g. C₅₋₇) cycloalkyl.

The term “heteroaryl” when used herein refers to an aromatic group containing one or more heteroatom(s) (e.g. one to four heteroatoms) preferably selected from N, O and S (so forming, for example, a mono-, bi-, or tricyclic heteroaromatic group). Heteroaryl groups include those which have between 5 and 14 (e.g. 10) members and may be monocyclic, bicyclic or tricyclic, provided that at least one of the rings is aromatic. However, when heteroaryl groups are bicyclic or tricyclic, they are linked to the rest of the molecule via an aromatic ring. Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Particularly preferred heteroaryl groups include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, thiophenyl, pyranyl, carbazolyl, acridinyl, quinolinyl, benzoimidazolyl, benzthiazolyl, purinyl, cinnolinyl and pterdinyl. Particularly preferred heteroaryl groups include monocylic heteroaryl groups.

For the avoidance of doubt, references herein to compounds of formula Ia or Ib include, where the context permits, references to any of compounds of formula Ia or Ib. Further, references to any of compounds of formula Ia or Ib include references to such compounds per se, to tautomers of such compounds, as well as to pharmaceutically acceptable salts or solvates, or pharmaceutically functional derivatives of such compounds.

Further embodiments of the invention that may be mentioned include those in which the compound of formula Ia or Ib is isotopically labelled. However, other particular embodiments of the invention that may be mentioned include those in which the compound of formula Ia or Ib is not isotopically labelled.

The term “isotopically labelled”, when used herein includes references to compounds of formula Ia or Ib in which there is a non-natural isotope (or a non-natural distribution of isotopes) at one or more positions in the compound. References herein to “one or more positions in the compound” will be understood by those skilled in the art to refer to one or more of the atoms of the compound of formula Ia or Ib. Thus, the term “isotopically labelled” includes references to compounds of formula Ia or Ib that are isotopically enriched at one or more positions in the compound.

The isotopic labelling or enrichment of the compound of formula Ia or Ib may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I).

When the compound of formula Ia or Ib is labelled or enriched with a radioactive or nonradioactive isotope, compounds of formula Ia or Ib that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

In particular embodiments that may be mentioned herein, in formula Ia, X may be N and Y may be NH. In further embodiments that may be mentioned herein, in Formula Ib, X may be NH and Y may be N.

In embodiments of the invention that may be mentioned herein:
(ai) R₁ may be a methyl group at the 6-position of the pyrimidine ring;
(aii) R₂ may be an ethyl group or a —CH₂COOCH₂CH₃ group;
(aiii) R₃ may be an aryl group. For example, the aryl group may be phenyl. Optionally, the aryl group may be substituted by one or more halogen atoms.

In embodiments of the invention that may be mentioned herein, the F₁F₀-ATP synthase inhibitor or a pharmaceutically acceptable salt or solvate thereof that selectively binds to the F₁ domain of the F₁F₀-ATP synthase is selected from the group consisting of:

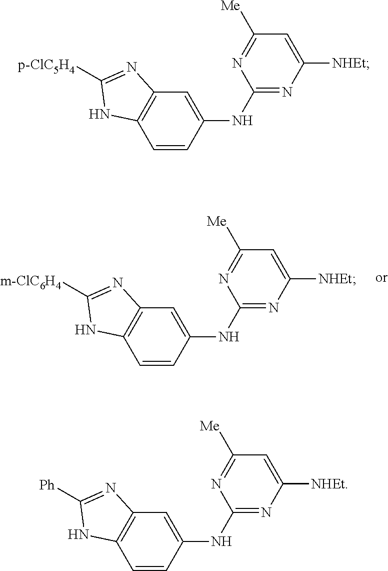

As will be appreciated, any suitable combination of the active ingredients set out herein may be used. Thus, in embodiments of the invention the combination may comprise:
(bi) a F₁F₀-ATP synthase inhibitor or a pharmaceutically acceptable salt or solvate thereof that selectively binds to the F₁ domain of the F₁F₀-ATP synthase and an NADH dehydrogenase inhibitor or a pharmaceutically acceptable salt or solvate thereof;
(bii) a F₁F₀-ATP synthase inhibitor or a pharmaceutically acceptable salt or solvate thereof that selectively binds to the F₁ domain of the F₁F₀-ATP synthase and a cytochrome-bcc:aa₃ inhibitor or a pharmaceutically acceptable salt or solvate thereof;
(biii) a F₁F₀-ATP synthase inhibitor or a pharmaceutically acceptable salt or solvate thereof that selectively binds to the F₁ domain of the F₁F₀-ATP synthase and a F₁F₀-ATP synthase inhibitor or a pharmaceutically acceptable salt or solvate thereof that selectively binds to the F₀ domain of the F₁F₀-ATP synthase;
(biv) a F₁F₀-ATP synthase inhibitor or a pharmaceutically acceptable salt or solvate thereof that selectively binds to the F₁ domain of the F₁F₀-ATP synthase, an NADH dehydrogenase inhibitor or a pharmaceutically acceptable salt or solvate thereof and a cytochrome-bcc:aa₃ inhibitor or a pharmaceutically acceptable salt or solvate thereof;
(bv) a F₁F₀-ATP synthase inhibitor or a pharmaceutically acceptable salt or solvate thereof that selectively binds to the F₁ domain of the F₁F₀-ATP synthase, an NADH dehydrogenase inhibitor or a pharmaceutically acceptable salt or solvate thereof and a F₁F₀-ATP synthase inhibitor or a pharmaceutically acceptable salt or solvate thereof that selectively binds to the F₀ domain of the F₁F₀-ATP synthase;
(bvi) a F₁F₀-ATP synthase inhibitor or a pharmaceutically acceptable salt or solvate thereof that selectively binds to the F₁ domain of the F₁F₀-ATP synthase, a cytochrome-bcc:aa₃ inhibitor or a pharmaceutically acceptable salt or solvate thereof; and a F₁F₀-ATP synthase inhibitor or a pharmaceutically acceptable salt or solvate thereof that selectively binds to the F₀ domain of the F₁F₀-ATP synthase;
(bvii) a F₁F₀-ATP synthase inhibitor or a pharmaceutically acceptable salt or solvate thereof that selectively binds to the F₁ domain of the F₁F₀-ATP synthase, an NADH dehydrogenase inhibitor or a pharmaceutically acceptable salt or solvate thereof, a cytochrome-bcc:aa₃ inhibitor or a pharmaceutically acceptable salt or solvate thereof, and a F₁F₀-ATP synthase inhibitor or a pharmaceutically acceptable salt or solvate thereof that selectively binds to the F₀ domain of the F₁F₀-ATP synthase.

Any suitable NADH dehydrogenase inhibitor or a pharmaceutically acceptable salt or solvate thereof may be used herein. For example, the NADH dehydrogenase inhibitor or a pharmaceutically acceptable salt or solvate thereof may be clofazimine.

Any suitable cytochrome-bcc:aa₃ inhibitor or a pharmaceutically acceptable salt or solvate thereof may be used herein. For example, the cytochrome-bcc:aa₃ inhibitor or a pharmaceutically acceptable salt or solvate thereof may be Q203:

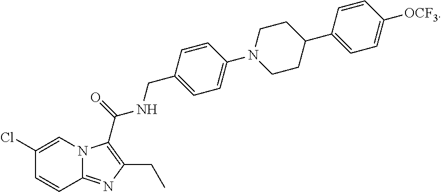 [SMILES: CCc1nc2ccc(Cl)cn2c1C(=O)NCc1ccc(N2CCC(c3ccc(OC(F)(F)F)cc3)CC2)cc1]

The combination disclosed herein may be presented as a single pharmaceutical formulation (containing all of the active ingredients in one formulation) or as a kit of parts comprising two or more pharmaceutical formulations (each formulation containing at least one active ingredient).

Thus, in aspects of the invention, there is provided:
(ci) a pharmaceutical composition comprising a pharmaceutically acceptable diluent or carrier and a combination as described hereinbefore (e.g. as set out in (bi) to (bvii) above); and
(cii) a kit of parts comprising a combination as described hereinbefore, and optionally, instructions for treating a patient. As noted above, the kit of parts may comprise a single formulation containing all of the active ingredients plus instructions for treating a patient or it may contain two or more pharmaceutical formulations, each containing at least one active ingredient to provide a combination as described in any one of (bi) to (bvii) above.

The combinations, the pharmaceutical compositions and kits of parts described herein may be useful in the treatment of subjects who are suffering from a bacterial infection. Therefore, in a further aspect of the invention, there is disclosed a use of a combination as described herein, a composition as described herein or a kit of parts as described herein in medicine.

In further aspects of the invention, there is disclosed:
(di) use of a combination as described herein, a composition as described herein or a kit of parts as described herein in the manufacture of a medicament to treat a bacterial infection;
(dii) a method of treating a bacterial infection comprising the steps of administering to a subject in need thereof a pharmaceutically acceptable amount of each component of the combination as described herein, wherein each component of the combination is administered sequentially, simultaneously or concomitantly with the other components;
(diii) a method of treating a bacterial infection comprising the steps of administering to a subject in need thereof a pharmaceutically acceptable amount of the composition as described herein;
(div) a method of treating a bacterial infection comprising the steps of administering to a subject in need thereof a pharmaceutically acceptable amount of each component of the kit of parts as described herein to a subject in need thereof, wherein each component of the kit of parts is administered sequentially, simultaneously or concomitantly with respect to each of the other components;
(dv) a combination as described herein, a composition as described herein or a kit of parts as described herein for use in the treatment of a bacterial infection.

In particular embodiments of the uses, methods, combinations for use, compositions for use and kit of parts for use, the bacterial infection may be tuberculosis.

Further aspects and embodiments of the invention are listed in the following numbered Statements.
1. A composition comprising:
   a) A compound of formula (Ia) or (Ib)

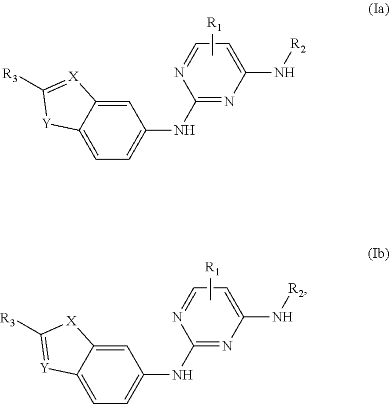

R₁ is hydrogen or a methyl group;
R₂ is an unsubstituted or substituted alkyl group;
R₃ is an aryl group or a heteroaryl group, optionally substituted by one or more groups selected from halogen, alkyl or alkoxy; and,
in Formula (Ia), X is CH or N and Y is NH, S or O, or,
in Formula (Ib), X is NH, S or O and Y is CH or N;
and
   b) One or more of Clofazimine, Q203 or TBAJ876.

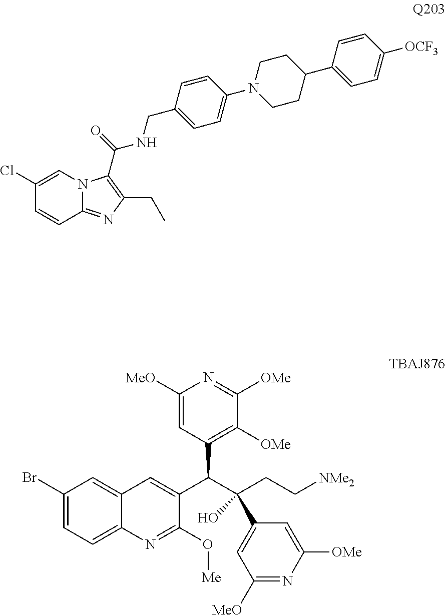 [SMILES: CCc1nc2ccc(Cl)cn2c1C(=O)NCc1ccc(N2CCC(c3ccc(OC(F)(F)F)cc3)CC2)cc1 ; COc1cc([C@](O)(CCN(C)C)[C@@H](c2cc3cc(Br)ccc3nc2OC)c2cc(OC)nc(OC)c2OC)cc(OC)n1]

2. The composition according to Statement 1, wherein, in Formula (Ia), X is N and Y is NH, or, in Formula (Ib), X is NH and Y is N.
3. The composition according to Statement 1 or 2, wherein R₁ is a methyl group at the 6-position of the pyrimidine ring.
4. The composition according to any of the preceding Statements, wherein R₂ is an ethyl group or a —CH₂COOCH₂CH₃ group.
5. The composition according to any of the preceding Statements, wherein R₃ is an aryl group.
6. The composition according to Statement 5, wherein the aryl group is substituted by one or more halogen atoms.
7. The composition according to any of the preceding Statements, wherein the compound of formula (Ia) or (Ib) is selected from the group consisting of:

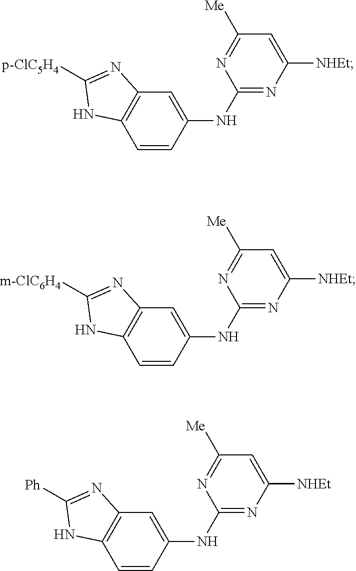

and tautomers thereof.
8. Use of a composition according to any one of Statements 1-7 in medicine.
9. A composition according to any one of Statements 1-7 for use in the treatment of a bacterial infection.
10. Use of a composition according to any one of Statements 1-7 in the manufacture of a medicament to treat a bacterial infection in a subject in need thereof.
11. A method of treating a subject suffering from a bacterial infection comprising the steps of administering to the subject a therapeutically effective amount of a composition according to any one of Statements 1-7, such that the bacterial infection is treated.
12. The composition according to Statement 9, the use according to Statement 10, or the method according to Statement 11, wherein the bacterial infection is tuberculosis.

Further aspects and embodiments of the invention will now be discussed by reference to the following non-limiting examples.

### EXAMPLES

### Materials

M. smegmatis mc² 155 (ATCC 35734) and M. bovis BCG (ATCC 700084) were gifted from from Prof. Thomas Dick lab. (Department of Microbiology and immunology, Yong Loo Lin School of medicine, National University of Singapore, Singapore). M. tuberculosis H37Rv was gifted from Prof. Thomas Dick lab. (Department of Microbiology and immunology, Yong Loo Lin School of medicine, National University of Singapore, Singapore). P. aeruginosa PAO1 WT, E. coli UTI 189 was gifted from Prof. Scott Rice, Singapore Centre for Environmental Life Sciences Engineering, Nanyang Technological University, Singapore. The cultures were maintained in complete Middlebrook 7H9 medium (Sigma-Aldrich) supplemented with 0.5% (vol/vol) glycerol (Promega), 0.05% Tween-80 (Sigma-Aldrich), and 10% Middlebrook albumin-dextrose-catalase (ADC) (Sigma-Aldrich). Corning T-25 mm² tissue culture flasks, 96-well clear flat bottom polystyrene microplates and 96-well white half area microplate were used for the experiments. Bacterial colonies were grown on Middlebrook 7H10 agar (Sigma-Aldrich) supplemented with 0.5% (vol/vol) glycerol (Promega), 0.05% Tween-80 (Sigma-Aldrich), and 10% Middlebrook oleic acid-albumin-dextrose-catalase (OADC) (Sigma-Aldrich). Bac titer glo and Cell titer glo were purchased from Promega. The following reagents like MOPS ((N-Morpholino) propane sulfonic acid), ADP (Adenosine diphosphate), NADH (Nicotinamide adenine dinucleotide), Succinic acid, Potassium phosphate, Clofazamine, DMSO (dimethyl sulfoxide), phosphate-buffered saline (PBS), ACMA (9-Amino-6-chloro-2-methoxyacridine), Thiorodazine, Tyrphostin A9, M9 salts, MgSO₄, CaCl₂), 0.4% w/v glucose, Terfenadine/tolbutamide, ACN, MeOH, CFZ were obtained from Sigma-Aldrich. Ananerobic atmosphere generation packets for oxygen consumption assays were purchased from Sigma. Bedaquiline (BDQ) and Telacebec (Q203) were purchased from MedChem Express. Compound TBAJ-876 was synthesized by Bioduro LLC (Beijing, China).

### General Procedure for Growing Bacteria

M. smegmatis mc² 155 and M. bovis BCG were cultured in complete 7H9 medium and grown to mid-exponential phase. Cells were inoculated to an initial optical density of 0.005. For broth culture, M. smegmatis was incubated at 37° C. under shaking at 80 rpm for 2-3 days in T-25 mm² tissue culture flasks. For M. bovis BCG, the culture was incubated at 37° C. under shaking at 80 rpm for 5 days in T-25 mm² tissue culture flasks. To count colony forming units (CFU ml-1), each culture was serially diluted in phosphate-buffered saline (PBS) (pH 7.0) and spotted on to agar plates.

### Preparation of GaMF1.39

GaMF1.39 was synthesized according to Hotra, A. et al., Angewandte Chemie Int. Ed. 2020, 59, 13295-13304.

To a suspension of 2,4-dichloro-6-methylpyrimidine (5.0 g, 30.7 mmol) in EtOH (50 mL) was added ethylamine hydrochloride (2.5 g, 30.7 mmol) and di-isopropylethylamine (2.5 eq.). The solution was heated at 50° C. for 24 hours before the reaction mixture was concentrated in vacuo to give a light yellow oil. The crude material was taken up in minimal amount of dichloromethane, adsorbed onto silica gel and purified by silica gel flash chromatography (0-100% EtOAc/Hexanes) to afford compound 2-chloro-N-ethyl-6-methylpyrimidin-4-amine as a light yellow solid (1.74 g, 33% yield). ¹H NMR (400 MHZ, CDCl₃) δ 6.06 (s, 1H), 5.06 (br. s, 1H), 3.32 (s, 2H), 2.33 (s, 3H), 1.24 (t, J=7.2 Hz, 3H); MS (ESI) m/z 172.1 [C₇H₁₀³⁵ClN₃+H]⁺, 174 (³⁷Cl M⁺+H⁺, 31); HRMS calcd for C₇H₁₁N₃³⁵Cl (M⁺+H⁺) 172.0642; found 172.0649 (4.1 PPM); FTIR (nujol, cm-1) vₘₐₓ 3250, 1600, 968; mp: 74-75° C.

A mixture of 4-chlorobenzoic acid (292 mg, 1.87 mmol) and 4-nitrobenzene-1,2-diamine (300 mg, 1.96 mmol, 1.05 eq) in polyphosphoric acid (4 mL) was stirred at 140° C. for 4 hours. The reaction was quenched by pouring into water (5 mL) and adjusted to pH 7 with 10 N NaOH solution. The precipitate was filtered and dried in vacuo to give a black residue. The crude product was purified by silica gel flash chromatography (0-40% EtOAc/Hexanes) to afford compound 2-(4-chlorophenyl)-5-nitro-1H-benzo[d]imidazole as a light green solid (142 mg, 28% yield). ¹H NMR (400 MHZ, DMSO-d₆) δ 13.7 (br. s, 1H), 8.49 (s, 1H), 8.23 (d, J=6.8 Hz, 2H), 8.14 (dd, J=8.8, 2.4 Hz, 1H), 7.78 (d, J=8.4 Hz, 1H), 7.69 (d, J=8.4 Hz, 2H); MS (ESI) m/z 274.0 [C₁₃H₈ClN₃O₂+H]⁺.

A mixture of 2-(4-chlorophenyl)-5-nitro-1H-benzo[d]imidazole (132 mg, 0.482 mmol), iron powder (269 mg, 4.82 mmol, 10 eq.) and NH₄Cl (258 mg, 4.82 mmol, 10 eq.) in 4:1 EtOH/water (5 mL) was heated at 80° C. for 5 hours. The mixture was filtered through a pad of celite, washing with MeOH. The filtrate was concentrated and the residue was taken up in water (10 mL) and extracted with EtOAc (3×5 mL). The combined organics were washed with brine, dried over Na₂SO₄, filtered and concentrated in vacuo give 2-(4-chlorophenyl)-1H-benzo[d]imidazol-5-amine as a brown oil (87.6 mg, 75% yield) which was used in the next step without further purification. ¹H NMR (400 MHZ, CDCl₃) δ 7.91 (d, J=8.4 Hz, 2H), 7.48-7.42 (m, 3H), 6.84 (s, 1H), 6.69 (dd, J=8.4, 2.0 Hz, 1H); MS (ESI) m/z 244.0 [C₁₃H₁₀ClN₃+H]⁺.

A solution of 2-chloro-N-ethyl-6-methylpyrimidin-4-amine (35.0 mg, 0.204 mmol) and 2-(4-chlorophenyl)-1H-benzo[d]imidazol-5-amine (74.5 mg, 0.306 mmol, 1.5 eq.) in n-BuOH (1 mL) was heated in a microwave reactor at 180° C. for 2 hours. The reaction mixture was azeotroped with toluene to give a white residue which was purified by preparative HPLC (20-50% MeCN/H2O; 0.1% formic acid) to afford desired compound (formate salt) as an off-white solid upon lyophilisation (12.0 mg, 15.5% yield). ¹H NMR (400 MHZ, DMSO-d₆) δ 12.6 (br. s, 1H), 9.00 (s, 1H), 8.28 (s, 1H), 8.14 (d, J=8.8 Hz, 2H), 7.59 (d, J=8.8 Hz, 2H), 7.45 (q, J=8.4 Hz, 2H), 6.98 (br. s, 1H), 5.78 (s, 1H), 3.37 (br. s, 2H), 2.14 (s, 3H), 1.18 (t, J=7.2 Hz, 3H); ¹³C NMR (100 MHZ, DMSO-d₆) δ 163.2, 163.0, 159.6, 133.8, 129.3, 129.1, 128.9, 127.8, 127.7, 34.9, 23.3, 14.7; MS (ESI) m/z 379.1 [C₂₀H₁₉ClN₆+H]⁺; HRMS (ESI) m/z calc'd for [C₂₀H₁₉ClN₆+H]⁺ 379.1433, found 379.1428; Melting point=142-143° C.

### Example 1. GaMF1.39 Targets Mycobacterial F-ATP Synthase

### Antimycobacterial Activity and Minimum Inhibitory Concentration (MIC 50) Determination

The growth inhibition dose-response assay was carried out using the broth microdilution method as described previously (Moreira, W., Aziz, D. B. & Dick, T., Front. Microbiol. 2016, 7, 199). Plates with the M. bovis BCG strain were incubated at 37° C. on an orbital shaker set at 80 rpm for 5 days. At the end of the incubation period, the culture in all wells was manually resuspended and the OD₆₀₀ was read using a TECAN Infinite Pro 200 plate reader. The MIC₅₀ reported represents the concentration that inhibits 50% of growth compared to drug free control.

### Production of IMVs, ATP Synthesis and ATP-Driven Proton Translocation Using IMVs

Preparation of M. smegmatis IMVs, ATP synthesis and ATP-driven proton translocation assays were performed according to Hotra, A. et al., FEBS J. 2016, 283, 1947-1961.

### Reconstitution and ATP Synthesis of Mycobacterial F-ATP Synthase

Recombinant mycobacterial F-ATP synthase was reconstituted according to Harikishore, A. et al., ACS Chemical Biol. 2022, 17, 529-535. For inhibitor studies with GaMF1.39 (90- and 180 nM) or BDQ (4 nM), proteoliposomes containing the reconstituted F-ATP synthase were additionally preincubated for 10 min at 4° C. with the respective inhibitor, before the ATP synthesis measurements were carried out.

Methylene blue assay Aliquots of 1.5 ml (OD₆₀₀=0.3), log-phase cultures of M. bovis BCG were transferred into clear 2 ml screw-cap glass vials along with test compounds. Thiorodazine was used as a positive control. The cultures were incubated in the presence of the drugs for 96 h at 37° C. before the addition of methylene blue dye at a final concentration of 0.001%. Upon addition of methylene blue, all vials were tightly closed and incubated at 37° C. in a hypoxic jar. Oxygen in the jar was removed by Anaerobic atmosphere Generation (AnaeroGen) sachet. The dye decolorizes when the oxygen in the culture is used up. The color changes and the differences in the exponentially growing cultures and broth reflect the relative oxygen consumption, thus indirectly indicate the respiration of each culture.

### Assay for ATP-Driven Proton Translocation

ATP-driven proton translocation into IMV's of M. smegmatis was measured by a decrease of 9-amino-6-chloro-2-methoxyacridine (ACMA) fluorescence using a Cary Eclipse

Fluorescence spectrophotometer (Varian Inc., Palo Alto) according to Haagsma, A. C. et al., FEMS microbiology letters, 2010, 313, 68-74. IMV's (0.18 mg per ml) were preincubated at 37° C. in 10 mM HEPES-KOH (pH 7.5), 100 mM KCl, 5 mM MgCl₂ containing 2 μM ACMA and a baseline was monitored for 5 min. The reaction was started by adding 2 mM ATP, or 2 mM NADH. After about 8 min, any proton gradient was collapsed by the addition of 2 μM of the uncoupler SF6847. The excitation and emission wavelengths were 410 nm and 480 nm, respectively.

### Results and Discussion

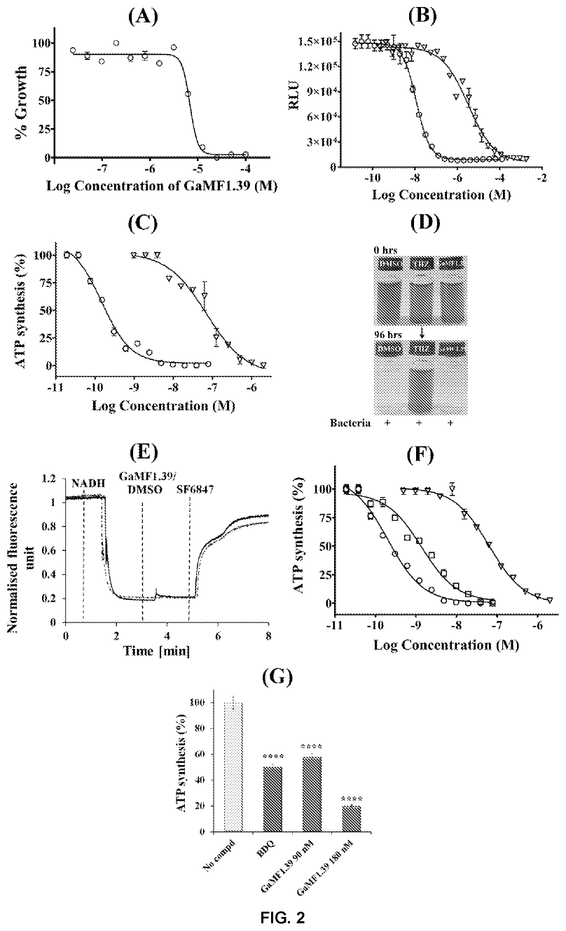

As shown by the minimal inhibitory concentration (MIC₅₀) of 6.8 μM (FIG. 2A), GaMF1.39 is a comparably efficacious inhibitor of M. bovis BCG and Mtb (3.0 μM) (Hotra, A. et al., Angewandte Chemie Int. Ed. 2020, 59, 13295-13304). The IC₅₀ of 3.3 μM measured for intracellular ATP inhibition of M. bovis BCG (FIG. 2B) was consistent with the determined MIC₅₀ and indicates that ATP formation, mainly synthesized by OXPHOS, is affected by the compound. NADH-driven ATP synthesis of IMVs of M. bovis BCG and M. smegmatis was inhibited at an IC₅₀ of 51.6±1.35 nM and 90±1.1 nM (Hotra, A. et al., Angewandte Chemie Int. Ed. 2020, 59, 13295-13304), respectively. In comparison, GaMF1.39 reduced ATP synthesis of IMVs in the presence of succinate with a similar IC₅₀=71 nM (FIG. 2C), indicating that the compound does not interfere with NADH-dehydrogenases. Oxygen consumption within the respiratory chain of M. bovis BCG is unaffected at 7 μM of GaMF1.39 (FIG. 2D), and GaMF1.39 lacked the ability to induce uncoupled proton pumping (FIG. 2E) unlike the known uncoupler SF6847, underscoring that GaMF1.39 does not induce an increase in respiration as described for the anti-TB drug BDQ (Lamprecht, D. A. et al., Nat. Commun. 2016, 7, 12393). In addition, we tested the potency of GaMF1.39 against M. smegmatis IMVs bearing an 166M substitution in subunit c, which is associated with resistance to BDQ (Kundu, S. (2017) Bedaquiline targets the epsilon subunit of mycobacterial F-ATP synthase. PhD thesis, National University of Singapore). 166M mutant IMVs displayed a 10-fold shift in sensitivity to BDQ with an IC₅₀ value of 1 nM in comparison to WT (IC₅₀ value of 0.14 nM) (FIG. 2F), whereas the mutation did not affect the potency of GaMF1.39, indicated by the IC₅₀ value of 64 nM. These data demonstrate that GaMF1.39 shows no cross resistance to BDQ.

Since GaMF1.39 lacked the ability to induce uncoupled proton pumping (FIG. 2E) and does not alter the membrane potential, we reconstituted the recombinant MsF-ATP synthase into proteoliposomes to confirm that this molecular engine is indeed the target of the molecule. The advantage of this reconstitution design for compound screening lies not only in its efficiency but also in the fact that it does not need a second proton-motive force generating enzyme, essential for target characterization. The reconstituted MsF-ATP synthase had an ATP synthesis activity of 35.2±2.3 nmol·min⁻¹ (mg protein)⁻¹ (FIG. 2G). When the control compound BDQ was used at its IC₅₀ concentration determined in IMVs (FIG. 2G), a ˜51% inhibition was observed, confirming that the engine is indeed BDQ's target, suggesting that the assay is a useful tool for demonstrating compound target specificity. Similarly, when GaMF1.39 was tested against the reconstituted MsF-ATP synthase at its IC₅₀ concentration, a 42% inhibition (20.5±2.6 nmol·min⁻¹ (mg protein)⁻¹) was calculated (FIG. 2G) and increasing the amount of GaMF1.39 resulted in a clear concentration dependent inhibition of the enzyme, underlining that the compound targets the enzyme.

### Example 2. GaMF1.39 is Bactericidal

Bacterial Killing Assay of M. smegmatis mc² 155

M. smegmatis mc² 155 cultures were grown to exponential phase and diluted to an OD₆₀₀ Of 0.005 and aliquoted onto T-25 mm² tissue culture flasks. Test compounds were dispensed into each flask and were incubated at 37° C. with shaking at 180 rpm for 5 days. Approximately 10 μl of culture was taken out from each flask, followed by the serial dilution with PBS. 25 μl of cultures of respective dilutions were plated on each quadrant of 7H10 agar plates. The agar plates were incubated at 37° C. for 3 days. Bacterial viability was determined by counting the colony-forming units (CFU).

Bacterial Killing Assay of M. bovis BCG

M. bovis BCG cultures were grown to exponential phase and diluted to an OD₆₀₀ of 0.005 and aliquoted onto T-25 mm² tissue culture flasks. Test compounds were dispensed into each flask and were incubated at 37° C. with shaking at 110 rpm for 6 days. Approximately 10 μl of culture was taken out from each flask, followed by the serial dilution with PBS. 25 μl of cultures of respective dilutions were plated on each quadrant of 7H10 agar plates. The agar plates were incubated at 37° C. for 10 days. Bacterial viability was determined by counting the colony-forming units (CFU).

### Toxicity Testing Using Zebrafish Embryos

Zebrafish care and ethics statement: All Zebrafish experiments were approved by the NTU Institutional Animal Care and Use Committee under reference no #A20038. Experiments were done using wild-type AB zebrafish. The ages of the embryos are shown in hours post fertilization (hpf).

Dechorionated zebrafish wild-type embryos at 48 hpf (n=120) were used. The embryos were immersed respectively in 5 ml of E3 medium containing GaMF1.39 in 6-well plate for 5 days. DMSO was used as vehicle control. The embryos were observed under a microscope. Abnormal phenotypes and survival rate from each treatment group were observed for 5 sequential days. The experiment was repeated twice with three replicates each.

### Biofilm Testing

Biofilm assay was performed in a 24-well plate to evaluate the activity of BDQ and GaMF1.39 on P. aeruginosa PAO1 WT and E. coli UTI 189 biofilms. Overnight cultures of P. aeruginosa PAO1 WT and E. coli UTI 189 were diluted in M9 glucose media (1×M9 salts, 2 mM MgSO₄, 0.1 mM CaCl₂), 0.4% w/v glucose) to a final OD₆₀₀ of 0.05. 1 ml of the diluted culture was then added into each well of the 24-well plate and incubated at 37° C. with 100 rpm shaking. Following 3 h of incubation, each well was washed once, and the culture media was replaced with 1 ml of 1×PBS (pH 7.4) or fresh M9 glucose medium containing 1-3 μM of BDQ or 3-9 μM of GaMF1.39. The treated samples were further incubated under the same conditions for 3 h. At t=6 h, the samples were collected. The 1×PBS (pH 7.4) buffer containing suspended bacteria cells with treatment was collected into 1.5 ml Eppendorf tubes and considered to be “planktonic samples”. Subsequently, each well was washed once with 1 ml 1×PBS (pH 7.4) before resuspending biofilm cells in the same volume of 1×PBS (pH 7.4). Biofilm cells were dislodged into the buffer by means of a cell scraper and 1 ml of the sample was collected into 1.5 ml Eppendorf tubes and labelled “biofilm samples”. The samples contained in Eppendorf tubes were sonicated in a water bath using the following settings: 5 min degas mode, 37 Hz, 100%, followed by 5 min pulse mode, 37 Hz, 100%. Subsequently, the samples were serially diluted and used for CFU counts. The experiment was repeated independently at least two times, with two technical replicate per independent experiment. CFU counts were analyzed using Graphpad Prism V9.3.0 using 2-way ANOVA and multiple comparison of column effect (concentration of compound against untreated control) within each row (planktonic vs biofilm samples).

### Microsomal Stability Assay

Microsomal stability assay were outsourced and performed by Bioduro-Sundia Ltd, USA based on the following protocol as done in Obach, R. S., Drug Metab. Dispos., 1999, 27, 1350; McGinnity, D. F. & Riley, R. J., Biochem. Soc. Trans., 2001, 29, 135. Working solutions of each compound are prepared from 10 mM stock solution in DMSO diluted to a final concentration of 100 μM in 0.05 M phosphate buffer (pH 7.4). Aliquots of Liver Microsome working solution are transferred into 1.1 mL tubes using a multichannel pipette. Positive control (5 mixed) and test compound working solutions are transferred into the tubes. The mixtures are vortexed gently and then pre-incubated at 37° C. 5 mM NADPH were aliquoted into the tubes using a multichannel pipette and vortexed gently. At each time point of 0 min, 5 min, 15 min, 30 min and 60 min with NADPH or 0, 30 min and 60 min without NADPH, an aliquot is removed from each tube. Terfenadine/tolbutamide in ACN/MeOH (1:1, v/v) is added to quench and precipitate the microsomal incubations. Samples are capped and vigorously vortexed and then centrifuged at 4° C. An aliquot of each supernatant is transferred for LC-MS/MSC analysis. The MS detection is performed by using a SCIEX API 4000 Q trap instrument. Each compound is analyzed by reversed phase HPLC using a Kinetex 2.6μ C18 100 Å column (3.0 mm×30 mm, Phenomenex). Mobile phase—Solvent A: water with 0.1% formic acid, solvent B: ACN with 0.1% formic acid. The amount of parent compound is determined on the basis of the peak area ratio (compound area to IS area) for each time point. The estimation of Clᵢₙₜ (in μL/min/mg protein) is calculated using the following equation: CLᵢₙₜ (μLmin⁻¹ mg⁻¹)=ln(2)*1000/t_{1/2}/protein concentration

The cLogP values were calculated using ChemDraw software Version 22.

### Results and Discussion

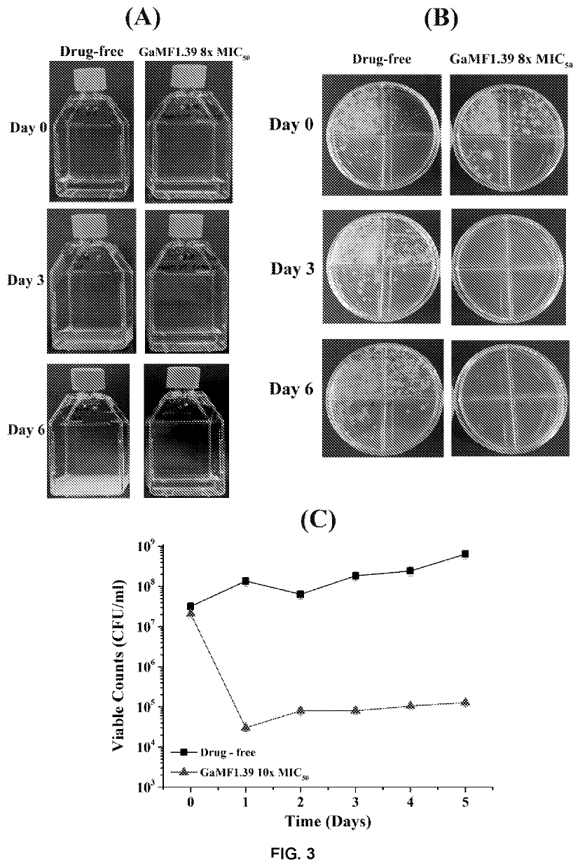

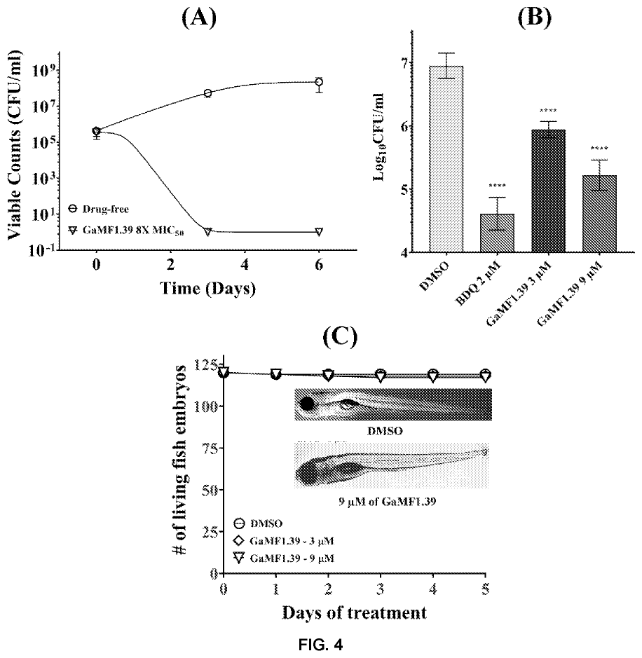

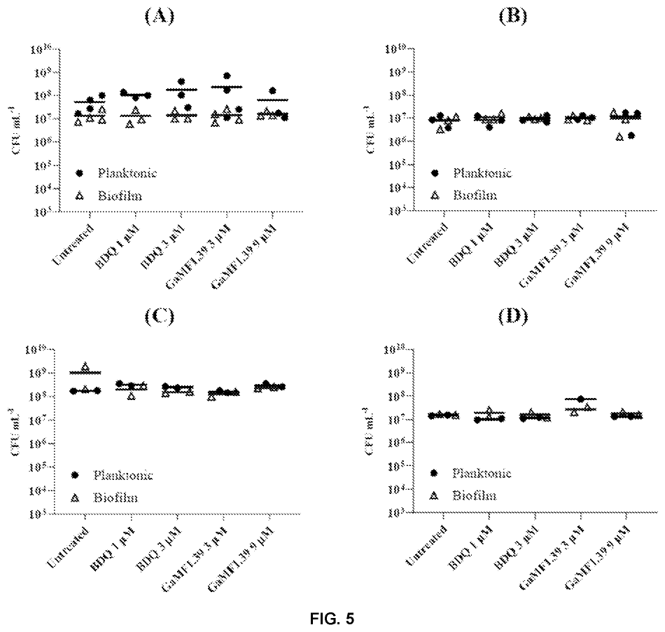

The killing efficiency of GaMF1.39 against M. bovis BCG was tested at 8-fold its MIC₅₀, which is shown clearly in flasks and on 7H10 agar plates (FIGS. 3A-B). At that concentration, GaMF1.39 (FIG. 4A) is more efficient than its parental molecule GaMF1 (Hotra, A. et al., Angewandte Chemie Int. Ed. 2020, 59, 13295-13304) and did not show delayed bactericidal activity like BDQ as reported before (Saw, W. G. et al., Scientific Rep. 2019, 9, 16759). GaMF1.39 also killed Mycobacterium smegmatis mc² 155 as well at 10-fold its MIC₅₀ (FIG. 3C).

To explore the anti-TB potency of the compound in macrophages, a THP-1 infection model was used. As shown in FIG. 4B, GaMF1.39 was active against M. tuberculosis H37Rv at 3- and 9 μM and showed a decrease in viable bacterial count (CFU/ml), when compared with initial inoculum (day 0). The infected macrophages did not show any visible optical alteration of the membrane, cell form or cell size as tested microscopically. This is in line with experiments performed on zebrafish embryos, where no signs of toxicity-induced killing were recorded in the presence of 9 μM of GaMF1.39 (FIG. 4C). Similarly, no killing of planktonic or biofilm cells were observed upon addition of 3-9 μM of GaMF1.39 to batch biofilms of Pseudomonas aeruginosa (P. aeruginosa) PAO1 WT or Escherichia coli (E. coli) UTI 189 under nutrient limited conditions, simulated by treatment in phosphate buffered saline (PBS), and under growth conditions, simulated by the addition of fresh growth medium (FIGS. 5A-D). These results confirm GaMF1.39's target specificity and indicates that the compound is not a broad-spectrum antibacterial. A microsomal stability assay showed that GaMF1.39 is metabolic stable in mouse liver microsomes (T_{1/2} of 29.6 min, Clᵢₙₜ of 46.8 ml/min/mg protein) while being slightly less lipophilic (clogP=6.51) than BDQ (clogP=7.25).

Here, we present a detailed antimycobacterial activity study of the bactericidal GaMF1.39 analogue, evidence for its specific targeting of the mycobacterial F-ATP synthase, and anti-TB potency in macrophages, without altering biofilm formation or being toxic to zebrafish larvae.

### Example 3. Compound Combinations Inhibit the OXPHOS-Pathway

### Checker-Board Titration Assay

A checkerboard titration assay was carried out as described previously (Hsieh, M. H. et al., Diagn. Microbiol. Infect. Dis. 1993, 16, 343-349; and Kaushik, A. et al., Antimicrob. Agents Chemother. 2015, 59, 6561-6567). Briefly, GaMF1.39 which was synthesized according to Hotra, A. et al., Angewandte Chemie Int. Ed. 2020, 59, 13295-13304, Q203, and CFZ, respectively, were added to complete 7H9 medium-containing 96-well flat-bottom Costar cell culture plates. Two-fold serial dilutions were done to allow 10 different concentrations of GaMF1.39 (0.1 μM to 60 μM) to be tested for interaction with 14 different concentrations Q203 (0.4 pM to 8 nM). Hence, a total of 140 different concentration combinations were tested between GaMF1.39 and Q203. In combination studies with CFZ, 10 different concentrations of GaMF1.39 (0.1 μM to 60 μM) were tested with 7 different concentrations of CFZ (0.03 μM to 2 μM). Each 96-well plate had a 7H9 medium-only control well and a drug-free bacterial culture control well. M. bovis (BCG) was cultured in complete 7H9 medium and grown to mid-exponential phase. Subsequently, the culture was diluted to an OD₆₀₀ of 0.01 using complete 7H9 medium and added to each well in the 96-well plate to create a final OD₆₀₀ value of 0.005. The plates were incubated for 6 days at 37° C. After the incubation period, the culture in each 96-well plate was manually resuspended, and the OD₆₀₀ of each well was read using a Tecan Infinite Pro 200 plate reader. Calculation of the fractional inhibitory concentration index (FICI) was done to analyze the results. The FICI is calculated as (MIC of drug A in combination/MIC of drug A alone)+ (MIC of DARQ B in combination/MIC of DARQ B alone). This calculation was done only for wells which showed 50% inhibition of bacterial culture growth compared to drug-free bacterial culture wells. A FICI of ≤0.5 indicates synergy, a FICI of >0.5 to 4 indicates additivity (no interaction), and a FICI of >4 indicates antagonism (Odds, F. C., J. Antimicrob. Chemother. 2003, 52, 1).

### Results and Discussion

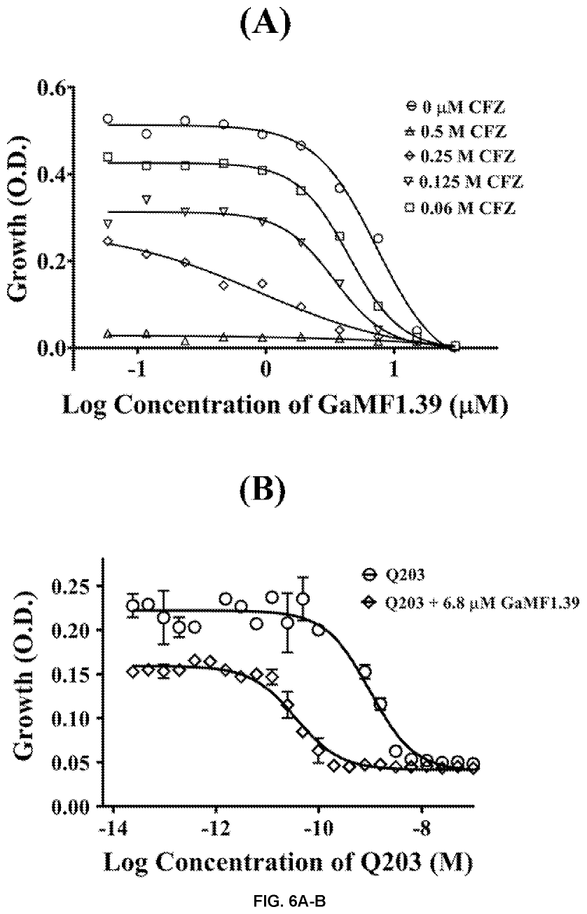

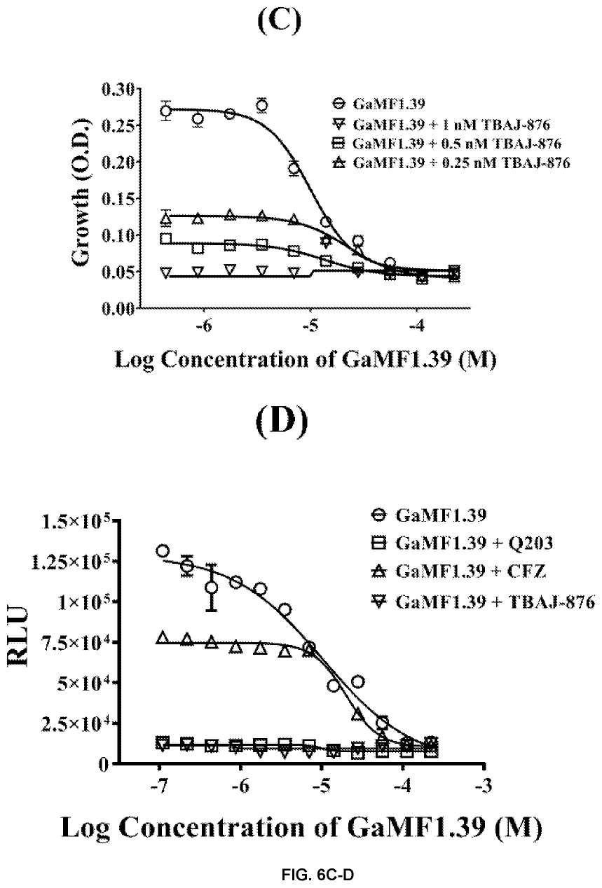

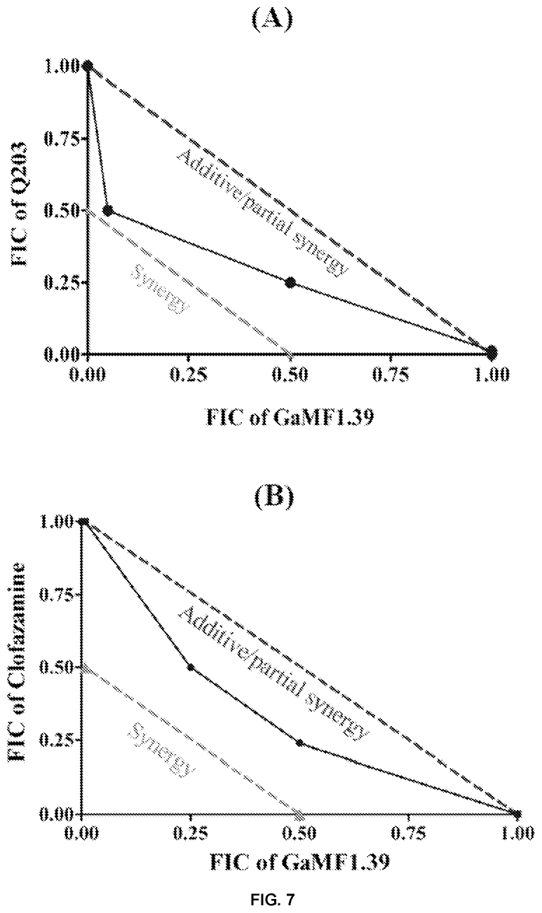

Here, we investigated the pharmacodynamic influence of GaMF1.39 with CFZ, effecting the first complex of the ETC. As revealed in FIG. 6A, CFZ reduced the MIC₅₀ of GaMF1.39 in a concentration dependent manner. The checkerboard assay demonstrates an additive/partial synergistic growth reduction triggered by the combination of CFZ and GaMF1.39 (FIG. 7A), achieving a minimum fractional inhibitory concentration (FIC) index of 0.75 in M. bovis BCG (Table 1). In comparison, the combination of the bc1 inhibitor Q203 and GaMF1.39 showed a significant growth reduction (FIG. 6B). The checkerboard assay revealed a slightly higher combinatory effect for GaMF1.39+Q203 compared to GaMF1.39+CFZ (as shown in FIG. 7B), and by the FIC index of 0.55 (Table 1). In addition, Q203 lowered the MIC₅₀ of GaMF1.39 from 6.8 μM to 0.23 μM (Table 1). Since GaMF1.39 showed no cross resistance to BDQ (see above), we tested whether GaMF1.39 would antagonize or potentiate the activity of the clinical development candidate and BDQ analogue TBAJ-876 which is less lipophilic (clogP=5.80), has higher clearance, and displays lower cardiotoxic potential than BDQ (Sutherland, H. S. et al., Bioorg. Med. Chem. 2019, 27, 1292-1307). As shown in FIG. 6C, GaMF1.39's potency was enhanced in the presence of different concentrations of TBAJ-876.

TABLE 1
The FICI was calculated as (MIC of drug A in combination/MIC of drug A alone) +
(MIC of drug B in combination/MIC of drug B alone) (Hsieh, M. H. et al., Diagn. Microbiol.
Infect. Dis. 1993, 16, 343-349). An FICI of ≤0.5 indicates synergy, a FICI of >0.5 to 4 indicates
additivity (no interaction), and an FICI of >4 indicates antagonism (Odds, F. C., J. Antimicrob.
Chemother. 2003, 52, 1).

| | | MIC | | | | | |
|---|---|---|---|---|---|---|---|
| | | | | Drug | | | |
| | Combination | Drug A | Drug A in | B | Drug B in | | |
| Drug A | with drug B | alone | combination | alone | combination | FICI | Outcome |
| GaMF1.39 | Q203 | 6.8 μM | 0.23 μM | 1 nM | 0.5 nM | 0.55 | Additivity |
| GaMF1.39 | Clofazamine | 6.8 μM | 3 μM | 0.25 μM | 0.125 μM | 0.75 | Additivity |

Growth inhibition of the regimen comprising GaMF1.39+Q203 or GaMF1.39+TBAJ-876 correlated well with the depletion of intracellular ATP formed in the presence of the combinations (FIG. 6D), underlining that the increased potency of the combinations was due to the reduction of OXPHOS. Interestingly, the decline of ATP formation of GaMF1.39+CFZ was moderate. Since CFZ decreases the central carbon metabolism and PMF, and generates reactive oxygen species (ROS) intracellularly (Lamprecht, D. A. et al., Nat. Commun. 2016, 7, 12393), the data imply that the enhanced potency is partly due to ATP synthesis inhibition but is also due to a reduced carbon metabolism and PMF as well as an increased formation of ROS.

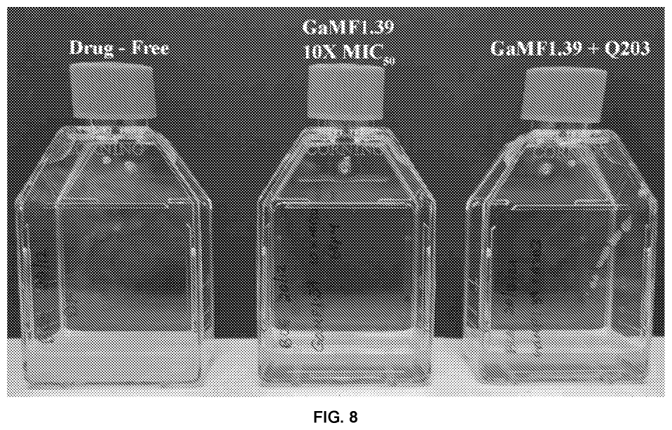

Because of the lower FIC index (0.55) of the GaMF1.39+Q203 compared to the GaMF1.39+CFZ combination, we tested whether the increase in growth reduction of the GaMF1.39+Q203 regimen (FIG. 6B) would also increase the killing potency of GaMF1.39 (FIG. 4A). FIG. 8 reveals that GaMF1.39 and GaMF1.39+Q203 inhibited the growth of M. bovis cells even after 10 days, showing that GaMF1.39 does not antagonize Q203 or increase the already existing killing potency of GaMF1.39.

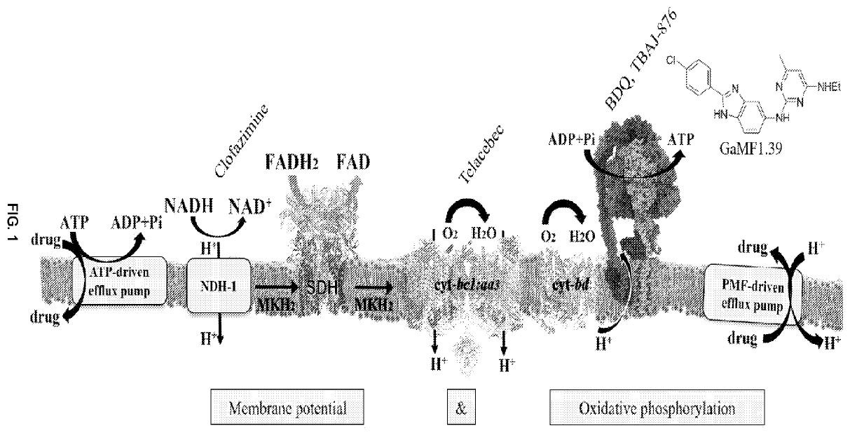

Taken together, the enzymes of the ETC are responsible for recycling of the reduced electron carriers NADH and FADH₂ from central carbon metabolism, thereby facilitating redox balance, generating a PMF essential for maintaining transmembrane electrochemical gradients to regulate PMF-driven pumps and to drive ATP synthesis via OXPHOS (FIG. 1) processes, which have been found to be required during persistence in Mtb (Gengenbacher, M. et al., Microbiology 2010, 156, 81-87; Rao, S. P. S. et al., Proc. Natl. Acad. Sci. U.S.A 2008, 105, 11945-11950; and Mackenzie, J. S. et al., Nat. Comm. 2020, 11, 6092). Both Q203 and BDQ have been reported to induce reduction in ATP levels by increasing central carbon metabolism, NADH production, oxygen consumption rate, and in the case of Q203, rerouting electron flux through cyt-bd oxidase, resulting in a bacteriostatic inhibition rather than killing activity (Lamprecht, D. A. et al., Nat. Commun. 2016, 7, 12393). A detailed study of BDQ's-induced cell death has revealed the rerouting of glycolysis via the pentose phosphate pathway back to the energy payoff phase of glycolysis to conserve energy and increase ATP formation by substrate-level phosphorylation, in line with the delayed cidal effect of BDQ (Saw, W. G. et al., Scientific Rep. 2019, 9, 16759; and Lamprecht, D. A. et al., Nat. Commun. 2016, 7, 12393). The bactericidal activity of BDQ is indistinguishable from that of the BDQ analogue TBAJ-876, and not effected by any uncoupling effect of both compounds (Sarathy, J. et al., Antimicrob. Agents Chemother. 2020, 64, e01540-19). By targeting the mycobacterial F-ATP synthase, GaMF1.39 depletes cellular ATP formation, required for replication, ana- and catabolic processes, cell wall formation and ATP-dependent efflux pumps, which play a central role in resistance by expelling the respective prodrug or drug (FIG. 1). The compound has the advantage that it does not affect oxygen consumption or H⁺-coupling and specifically inhibits the mycobacterial F-ATP synthase from generating ATP. Drug-drug potency interaction studies with the ETC- or OXPHOS inhibitors CFZ, Q203 and TBAJ-876 revealed no antagonistic effect, which is a prerequisite for combinatory approaches to treat Mtb infections. Hence, GaMF1.39 potentiates the anti-TB activity of the NADH Dehydrogenase (NDH) inhibitor CFZ, the cyt-bcc:aa₃ inhibitor Telacebec (Q203) alone, as well as TBAJ-876, respectively. Furthermore, GaMF1.39's efficacy increased in combination with CFZ, Q203 or TBAJ-876 so there is enhanced whole ATP synthesis inhibition and anti-tuberculosis activity. These results suggest that GaMF1.39 may add value to a compound-combination targeting oxidative phosphorylation for tuberculosis treatment. GaMF1.39 is potent in cell death and against Mtb infected macrophages without effecting biofilm formation or Zebrafish embryonic development or viability, opening the door for efficacy studies in Mtb infected mouse models, and paves the way for new combinatory approaches for the treatment of MDR-TB and XDR-TB.

1. A combination comprising:
(i) a F₁F₀-ATP synthase inhibitor or a pharmaceutically acceptable salt or solvate thereof that selectively binds to the F₁ domain of the F₁F₀-ATP synthase; and
one or more of the compounds selected from:
(ii) an NADH dehydrogenase inhibitor or a pharmaceutically acceptable salt or solvate thereof;
(iii) a cytochrome-bcc:aa₃ inhibitor or a pharmaceutically acceptable salt or solvate thereof; and
(iv) a F₁F₀-ATP synthase inhibitor or a pharmaceutically acceptable salt or solvate thereof that selectively binds to the F₀ domain of the F₁F₀-ATP synthase, provided that the F₁F₀-ATP synthase inhibitor or a pharmaceutically acceptable salt or solvate thereof that selectively binds to the F₀ domain of the F₁F₀-ATP synthase is not diarylquinoline bedaquiline.

2. The combination according to claim 1, wherein the F₁F₀-ATP synthase inhibitor that selectively binds to the F₁ domain of the F₁F₀-ATP synthase is a compound of formula Ia or Ib: 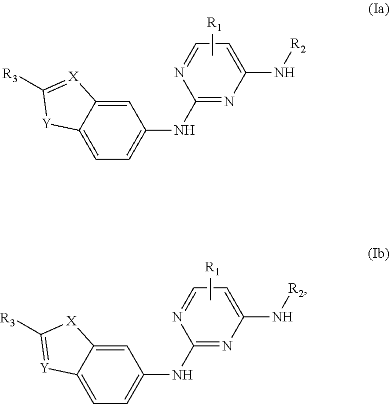
where:
R₁ is hydrogen or a methyl group;
R₂ is an unsubstituted or a substituted alkyl group;
R₃ is an aryl group or a heteroaryl group that is unsubstituted or substituted by one or more groups selected from halogen, alkyl or alkoxy; and,
In Formula Ia, X is CH or N and Y is NH, S or O, or,
in Formula Ib, X is NH, S or O and Y is CH or N, or a pharmaceutically acceptable salt or solvate thereof.

3. The combination according to claim 2, wherein:
in Formula Ia, X is N and Y is NH; or
in Formula Ib, X is NH and Y is N.

4. The combination according to claim 2, wherein one or more of the following apply:
(a) R₁ is a methyl group at the 6-position of the pyrimidine ring;
(b) R₂ is an ethyl group or a —CH₂COOCH₂CH₃ group;
(c) R₃ is an aryl group.

5. (canceled)

6. (canceled)

7. (canceled)

8. The combination according to claim 1, wherein the F₁F₀-ATP synthase inhibitor or a pharmaceutically acceptable salt or solvate thereof that selectively binds to the F₁ domain of the F₁F₀-ATP synthase is selected from the group consisting of:

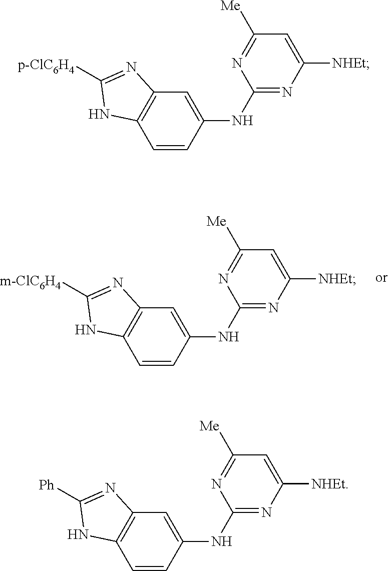

9. The combination according to claim 1, wherein the combination comprises a F₁F₀-ATP synthase inhibitor or a pharmaceutically acceptable salt or solvate thereof that selectively binds to the F₁ domain of the F₁F₀-ATP synthase and an NADH dehydrogenase inhibitor or a pharmaceutically acceptable salt or solvate thereof.

10. The combination according to claim 1, wherein the combination comprises a F₁F₀-ATP synthase inhibitor or a pharmaceutically acceptable salt or solvate thereof that selectively binds to the F₁ domain of the F₁F₀-ATP synthase and a cytochrome-bcc:aa₃ inhibitor or a pharmaceutically acceptable salt or solvate thereof.

11. The combination according to claim 1, wherein the combination comprises a F₁F₀-ATP synthase inhibitor or a pharmaceutically acceptable salt or solvate thereof that selectively binds to the F₁ domain of the F₁F₀-ATP synthase and a F₁F₀-ATP synthase inhibitor or a pharmaceutically acceptable salt or solvate thereof that selectively binds to the F₀ domain of the F₁F₀-ATP synthase.

12. The combination according to claim 1, wherein the combination comprises a F₁F₀-ATP synthase inhibitor or a pharmaceutically acceptable salt or solvate thereof that selectively binds to the F₁ domain of the F₁F₀-ATP synthase, an NADH dehydrogenase inhibitor or a pharmaceutically acceptable salt or solvate thereof and a cytochrome-bcc:aa₃ inhibitor or a pharmaceutically acceptable salt or solvate thereof.

13. The combination according to claim 1, wherein the combination comprises a F₁F₀-ATP synthase inhibitor or a pharmaceutically acceptable salt or solvate thereof that selectively binds to the F₁ domain of the F₁F₀-ATP synthase, an NADH dehydrogenase inhibitor or a pharmaceutically acceptable salt or solvate thereof and a F₁F₀-ATP synthase inhibitor or a pharmaceutically acceptable salt or solvate thereof that selectively binds to the F₀ domain of the F₁F₀-ATP synthase.

14. The combination according to claim 1, wherein the combination comprises a F₁F₀-ATP synthase inhibitor or a pharmaceutically acceptable salt or solvate thereof that selectively binds to the F₁ domain of the F₁F₀-ATP synthase, a cytochrome-bcc:aa₃ inhibitor or a pharmaceutically acceptable salt or solvate thereof; and a F₁F₀-ATP synthase inhibitor or a pharmaceutically acceptable salt or solvate thereof that selectively binds to the F₀ domain of the F₁F₀-ATP synthase.

15. The combination according to claim 1, wherein the combination comprises a F₁F₀-ATP synthase inhibitor or a pharmaceutically acceptable salt or solvate thereof that selectively binds to the F₁ domain of the F₁F₀-ATP synthase, an NADH dehydrogenase inhibitor or a pharmaceutically acceptable salt or solvate thereof, a cytochrome-bcc:aa₃ inhibitor or a pharmaceutically acceptable salt or solvate thereof, and a F₁F₀-ATP synthase inhibitor or a pharmaceutically acceptable salt or solvate thereof that selectively binds to the F₀ domain of the F₁F₀-ATP synthase.

16. The combination according to claim 1, 12, 13 and 15, wherein the NADH dehydrogenase inhibitor or a pharmaceutically acceptable salt or solvate thereof is clofazimine.

17. The combination according to claim 1, wherein the cytochrome-bcc:aa₃ inhibitor or a pharmaceutically acceptable salt or solvate thereof is Q203:

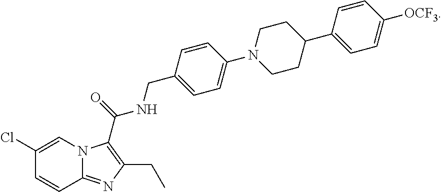 [SMILES: CCc1nc2ccc(Cl)cn2c1C(=O)NCc1ccc(N2CCC(c3ccc(OC(F)(F)F)cc3)CC2)cc1]

18. The combination according to claim 1, wherein the F₁F₀-ATP synthase inhibitor or a pharmaceutically acceptable salt or solvate thereof that selectively binds to the F₀ domain of the F₁F₀-ATP synthase is TBAJ876:

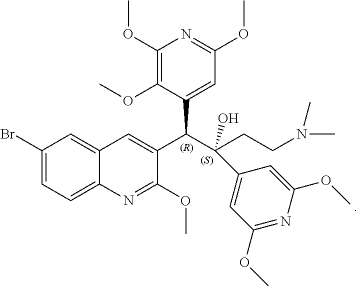

19. A pharmaceutical composition comprising a pharmaceutically acceptable diluent or carrier and a combination according to claim 1.

20. A kit of parts comprising a combination according to claim 1, and optionally, instructions for treating a patient.

21. (canceled)

22. (canceled)

23. A method of treating a bacterial infection comprising the steps of administering to a subject in need thereof a pharmaceutically acceptable amount of each component of the combination as described in claim 1, wherein each component of the combination is administered sequentially, simultaneously or concomitantly with the other components.

24. A method of treating a bacterial infection comprising the steps of administering to a subject in need thereof a pharmaceutically acceptable amount of the composition as described in claim 19.

25. A method of treating a bacterial infection comprising the steps of administering to a subject in need thereof a pharmaceutically acceptable amount of each component of the kit of parts as described in claim 20 to a subject in need thereof, wherein each component of the kit of parts is administered sequentially, simultaneously or concomitantly with respect to each of the other components.

26. (canceled)

27. (canceled)
